(19) United States
(12) Patent Application Publication
(10) Pub. No.: US 2004/0049155 A1
(43) Pub. Date: Mar. 11, 2004

(54) Needle tip protector

(76) Inventor: John B. Schramm, Skokie, IL (US)

Correspondence Address: Richard D. Harris, GREENBERG TRAURIG. P.C., Suite 2500, 77 West Wacker Drive, Chicago, IL, 60601, US

(21) Appl. No.: 10/425,356

(22) Filed: Apr. 29, 2003

Related U.S. Application Data
(60) Provisional application No. 60/386,947, filed on Jun. 6, 2002.

(51) Int. Cl. A61M 5/00

(52) U.S. Cl. 604/110; 128/919

(57) ABSTRACT

Needle tip protectors or guards, for preventing re-use of hypodermic or other needles, and/or accidental needle stick during the disposal thereof, are provided. The needle tip guard is mounted on the shaft of a needle, prior to use of the needle. The needle tip protectors or guards are provided with structures for binding or frictionally engaging against a needle shaft, after a needle has been used and the needle tip guard has been slid along the length of the used needle, until the tip of the needle is withdrawn into the interior of the needle tip guard. The needle tip protectors or guards are also provided with structures to prevent re-emergence of the needle tips out of the fronts of the guards, once the tips of the needles have been withdrawn into the guards.

This application claims the benefit under 35 U.S.C. 119(e) of the filing date of provisional application Ser. No. 60/366,947, filed Jun. 6, 2002.

### BACKGROUND OF THE INVENTION

1. Field of the Invention

The present invention relates in general to devices for covering and guarding the tip of a needle or other sharp medical instrument after use and, more particularly, to a needle tip guard which locks into place over the tip of a sharp needle after the needle has been used to prevent accidental puncture or reuse.

2. Background Art

Devices designed to protect the typically sharp tip of a needle have been known in the art for many years. In particular, given the wide concern regarding AIDS and other diseases that are carried in human blood and transmitted through the bloodstream, it has been highly desirable to provide protectors or guards to cover the tip of hypodermic needles, catheters, or other sharp medical needle-type devices after those devices have been used. Needle tip guards have been designed to fit over the tip of the needle after the needle has been used, to both guard against subsequent use, as well as to prevent inadvertent puncture. Reuse can result when used hypodermic or other needles are thrown into a garbage receptacle or recycling receptacle and subsequently recovered for unintended or illegal use. Likewise, spent needles in a disposable container may inadvertently puncture or penetrate a person reaching into such as container to dispose of used needles or other waste.

Needle safety technology has become even more prevalent in recent years as many states have passed needle safety laws with specific guidelines for sharps needle protection technology. In particular, those laws have specified that all sharp needle tips used in medical applications require some form of needle tip guard to prevent inadvertent injury and puncture by the needle tip, as well as to eliminate subsequent reuse of already used needles. Reuse is a particularly strong concern as illegal drug use often occurs with used needles.

In response to the used needle problems discussed above, as well as the recent legislation, there have been a number of needle guard products designed specifically to offer sharps protection by locking over the tip of a needle, after use of the needle. Several of those needle guards, including those shown and described in Kulli, U.S. Pat. No. 4,929,241; Chamuel, U.S. Pat. No. 5,053,017; Sircom, et al., U.S. Pat. No. 5,322,517; Sircom, U.S. Patent No. 5,662,610; and Caizza et al., U.S. Pat. No. 5,910,130, have taken a form of needle guards which are mounted and slide on the shaft of a needle. In particular, these guards include a locking latch or jaw-like mechanism that covers the tip of the needle when the needle guard is slid past the needle tip.

Kulli, U.S. Pat. No. 4,929,241, discloses a protective guard formed as a single, unitary clip which rides on the shaft of a needle. The protective guard includes a base portion and a pair of flexible jaws which extend directly from and distally away from the base portion. The base portion includes an aperture for mounting the needle, through which aperture the shaft of the needle is received. The flexible jaws are biased to converge toward one another, and each jaw has a shield portion extending substantially transversely to the portion of the jaw which extends along the needle shaft. The ends of each transverse shield portion directly contacts the shaft of the needle before deployment of the device. The edges of each of the shields may be provided with an arcuate, even semi-circular groove, for engaging the sides of the needle shaft, when the clip is in its undeployed orientation (i.e., when the needle tip is still exposed). The flexible jaws also include sharp blades which extend from opposing inner surfaces of the flexible jaws at locations between the base and the transverse shields. The blades do not contact the sides of the shaft until the needle has been withdrawn from between the shield edges, thus deploying the protective guard and enabling the legs to move toward one another and the shields to overlap, covering the tip of the needle. The edges of the blades thus engage the sides of the needle shaft, toward preventing the needle shaft from being completely withdrawn from the guard.

Chamuel, U.S. Pat No. 5,053,017 discloses yet another needle tip guard, in the style of a clip, formed, in a basic embodiment, from a single, unitary convoluted strip of metal or other flexible material. The guard includes a base portion and two legs extending distally therefrom, one leg extending along and adjacent to the top portion of the needle and the other leg extending along and adjacent to the bottom portion of the needle. The bottom leg has a guide portion to assist in slidably maintaining the guard on the needle shaft. The upper leg includes a needle shaft engaging end portion and an inwardly folded bight which is configured to frictionally engage the side of the needle shaft, once the point of the needle has been pulled from between the free ends of the strip. The ends of the legs are biased to retain the needle shaft between the ends of the strip prior to deployment, but to overlap one another upon retraction of the needle tip past the end portion of the upper leg. The base portion includes an aperture through which the needle passes prior to passing between the otherwise overlapping ends of the strip. The area around the aperture is likewise configured to deform and frictionally engage the needle upon deployment of the device. Additional embodiments of the Chamuel '017 reference employ biased rocking members which pivot, once the tip of the needle has been slid past a certain location, to block the re-emergence of the point, as well as to cause gripping engagement of the shaft of the needle.

The Sircom '517 and '610 references each disclose an independently spring biased, U-shaped latching member which is housed within a needle guard, and which is prompted over the needle tip after the needle tip is withdrawn past the latching member and into the needle guard. In one embodiment, the latching member includes a locking base plate (the longer leg of the “U”), a lever arm and a shield (the shorter leg of the “U”). The locking base plate includes an aperture through which the needle slides, which aperture digs into the shaft of needle upon canting of the latching member. The shield contacts the needle before retraction of the needle into the housing and deployment of the latching member. The housing further includes the spring biasing member which bears against the latching member to cant the latching member about a pivot point formed between the lever arm and the inner surface of the housing, upon retraction of the needle tip past the shield. Thus, upon deployment, the latching member prevents movement of the needle back out of the guard, while also digging into the shaft of the needle to resist movement of the needle guard off of the needle.

Other embodiments of these patents employ outer shells, through which the needles pass, when the needles are in their usable positions. Upon pulling the needle tips into the housings, the needles unobstruct various structures which are then permitted to pivot, under coil spring biasing, into positions in which the needle tips are precluded from exiting back through the fronts of the housings. Various clutching mechanisms are also disclosed for frictionally engaging the sides of the needle shaft, once the tip of the needle has been withdrawn into the housing.

Caizza, U.S. Pat. No. 5,910,130, discloses a needle guard in the form of a housing, through which the needle shaft passes. A sigma-shaped unitary spring clip is mounted within the housing, with the shaft passing through an aperture in one foot of the sigma, and the edge of the other foot of the sigma bearing against the side of the shaft. When the needle shaft is withdrawn, the spring foot of the shaft moves across and block the path of the needle tip. The reference also discloses a form of levered mechanism for moving the housing along the needle shaft, without bringing the user's fingers into the vicinity of the needle tip.

In use of these devices, the needle guard is first mounted onto the base of the needle shaft before the needle is used. Then, after the needle has been used, the guard is slid away from the base of the needle, toward the distal end of the needle and over the tip of the needle. Once the guard advances past the needle tip, it automatically locks into place on the distal end of the needle shaft, thus anchoring on the distal end of the needle and acting as a shield over the needle tip.

While these and other needle guards have worked well to prevent injury and reuse of hypodermic needles and other sharp-tipped medical devices, these prior art devices have been difficult to mount onto a given needle, without damaging the needle tip. In particular, mounting the needle guards over sharp needle tips may lead to needle puncture or other injury, particularly if the needle guards are placed on the needle shaft without proper instructions or mounting equipment. Accordingly, these needle guards typically must be mounted to the corresponding needle, catheter or other medical device by the manufacturer, thus preventing application of these needle guards to needles, catheters and other medical devices originally unadorned with a needle guard. It is believed that needle-type devices are still manufactured and sold without a needle guard.

It is thus desirable to provide a needle protector or guard which may be easily mounted onto the shaft of a hypodermic needle or other sharp catheter, biopsy needle or other needle-type medical device to allow use of the needle protector or guard with a number of needle-type devices.

It is likewise desirable to allow mounting of the needle protector or guard onto a given needle with relative ease, to minimize the time it may take for a needle user to mount a needle protector or guard onto to base of a needle.

It is also desirable to provide a needle guard carrier which may be placed interchangeably onto different types of needles to ensure that a user is able to lock the needle protector or guard over the tip of the needle after use to prevent injury or reuse.

These and other desirable characteristics of the invention will become apparent in view of the present specification, including the claims, and drawings.

### SUMMARY OF THE INVENTION

The present invention is directed in part to a needle tip guard for placement on the shaft of a hollow needle, for positioning over a tip region of the hollow needle, following completion of use of the hollow needle, for isolating the tip region of the hollow needle. The needle tip guard comprises, in part, a housing, operably configured to describe a void region and having a front end region and a rear end region. The housing has at least one passage therethrough operably configured to receive a shaft of a hollow needle therethrough. A resilient shield member is disposed within said void region within the housing and configured for movement between a first, undeployed position and a second deployed position, whereupon the positioning of a hollow needle shaft through the housing extending from the first end region to the second end region, the resilient shield member is retained in its first undeployed position, in a deformed configuration with a portion of the inserted hollow needle shaft preventing movement of the shield member into the deployed configuration; in which stored force in the deformed shield member, upon movement of an inserted hollow needle shaft relative to the housing such that a tip of the hollow needle shaft is received within the void region, prompts the shield member to reorient from the first position to the second deployed position. The resilient shield member, in the second deployed position, is disposed to simultaneously preclude re-emergence of a tip of the hollow needle shaft out from the front end region of the housing, and exert a restraining force against the hollow needle shaft toward inhibiting movement of the hollow needle shaft relative to the housing.

The needle tip guard may further comprise a needle shaft carrier, removably inserted into the housing, and extending from the front end region through to the rear end region thereof. The needle shaft carrier preferably has a hollow configuration with an inner passage which is configured to insertingly a hollow needle member, and is configured for facilitating insertion of a hollow needle shaft, and subsequent removal of the needle shaft guide member from the housing, prior to use of the hollow needle.

The shield member preferably has a first end and a second end, the first end having an aperture operably configured to receive a hollow needle shaft therethrough, the second end being disposed to bear against a side portion of a hollow needle shaft, when the shield member is in its first undeployed position. The second end is disposed in a path of movement of the hollow needle shaft, when the shield member is in its second deployed position, to preclude subsequent movement of a tip of the hollow needle shaft out of the front end region of the housing. The structure surrounding the aperture in the first end of the shield member is preferably operably configured to frictionally engage a hollow needle member extending therethrough, when the shield member is disposed in its second deployed position. The second end of the shield member is preferably configured as a straight edge region, configured to obliquely abut a side surface of a hollow needle shaft, when the shield member is in its first position. The second end of the shield member may alternatively be configured as a substantially U-shaped turned under region, a curved bight portion of which is configured to obliquely abut a side surface of a hollow needle shaft, when the shield member is in its second position.

The needle tip guard may further comprise an outer shell operably configured to enclose and receive the housing. At least one resilient attachment member may be disposed proximate the rear end region of the housing, with at least one attachment member engagement region disposed proximate a rear end region of the outer shell. Alternatively, at least one resilient attachment member may be disposed proximate the front end region of the housing and at least one attachment engagement region may be disposed proximate a front end region of the outer shell.

A guide lip, may be provided, extending from the second end of the shield member and disposed for sliding contact relative to a portion of the surface of a hollow needle member insertingly disposed within the housing, upon relative movement of the hollow needle member relative to the needle tip guard.

The shield member may have an end configured to bear against a side portion of a hollow needle shaft, when the shield member is in its first undeployed position. The end may be configured to move into a path of movement of the hollow needle shaft, when the shield member is in its second deployed position, to preclude subsequent movement of a tip of the hollow needle shaft out of the front end region of the housing. The void region within the housing may be defined in part by an interior forward wall, along which an end of the shield member is configured to slide. The interior forward wall may be straight. Alternatively, the interior forward wall has a first portion which is disposed at a first included angle, relative to the shaft of a hollow needle which has been inserted into the housing, which first portion is distal to the hollow needle shaft, and a second portion, proximate the hollow needle shaft, which is disposed at a second included angle, relative to the shaft of a hollow needle which has been inserted into the housing, which is less than the first included angle.

The invention is also directed in part to a needle tip guard for placement on the shaft of a hollow needle, for positioning over a tip region of the hollow needle, following completion of use of the hollow needle, for isolating the tip region of the hollow needle, wherein the needle tip guard comprises, in part, a housing, operably configured to describe a void region, and having a front end region and a rear end region. The housing has at least one passage therethrough operably configured to receive a shaft of a hollow needle therethrough. A support block has opposed first and second ends, and disposed within said void, and having a passage extending therethrough from the first end to the second end for receiving the shaft of a hollow needle. A shield member is disposed within the void and configured for pivotable movement within the void between an aligned configuration and an unaligned configuration. The shield member has a substantially rigid, C-shaped configuration, with first and second arm portions overlying the first and second ends of the support block, the first and second arm portions having apertures disposed therein, which align with the passage through the support block, when a hollow needle shaft is disposed in the passage and extending from the front end region through to the rear end region of the housing. A biasing member operably interconnects the shield member, the support block and the housing, for prompting the shield member from the aligned position to the unaligned position, upon withdrawal of a tip portion of the hollow needle shaft to a position within the void between the shield member and the support block. A clamp plate is operably associated with the shield member, for pivotable movement therewith, to frictionally engage the hollow needle shaft, upon movement of the shield member to the unaligned position, to resist withdrawal of the hollow needle shaft from the rear end region of the housing. The shield member, upon movement to the unaligned position, is disposed to preclude re-emergence of the tip portion of the hollow needle shaft from the front end region of the housing.

A needle shaft carrier may be removably inserted into the housing, and extending from the front end region through to the rear end region thereof, the needle shaft carrier having a hollow configuration with an inner passage which is configured to insertingly a hollow needle member. The needle shaft carrier is configured for facilitating insertion of a hollow needle shaft, and subsequent removal of the needle shaft guide member from the housing, prior to use of the hollow needle.

The present invention also comprises in part a needle tip guard for placement on the shaft of a hollow needle, for positioning over a tip region of the hollow needle, following completion of use of the hollow needle, for isolating the tip region of the hollow needle, wherein a housing is provided, operably configured to describe a void region, and having a front end region and a rear end region. The housing has at least one passage therethrough operably configured to receive a shaft of a hollow needle therethrough. A support block has opposed first and second ends, and disposed within said void, and having a passage extending therethrough from the first end to the second end for receiving the shaft of a hollow needle. A shield member is disposed within the void and configured for pivotable movement within the void between an aligned configuration and an unaligned configuration. The shield member has a substantially rigid, C-shaped configuration, with a planar web, and first and second arm portions extending therefrom, the first and second arm portions overlying the first and second ends of the support block, the first and second arm portions each having an aperture disposed therein, which apertures align with the passage through the support block, when a hollow needle shaft is disposed in the passage and extending from the front end region through to the rear end region of the housing. A biasing member operably interconnects the shield member, the support block and the housing, for prompting the shield member from the aligned position to the unaligned position, upon withdrawal of a tip portion of the hollow needle shaft to a position within the void between the shield member and the support block. The shield member, upon movement to the unaligned position, is disposed to preclude re-emergence of the tip portion of the hollow needle shaft from the front end region of the housing and to exert a resistive force against a side surface of the hollow needle shaft, toward inhibiting further movement of the hollow needle shaft relative to the needle tip guard.

The biasing member may comprise a spring member, mounted within the support block. Alternatively, the biasing member may comprise a spring member, mounted within the housing.

A needle shaft carrier may be removably inserted into the housing, and extending from the front end region through to the rear end region thereof, the needle shaft carrier having a hollow configuration with an inner passage which is configured to insertingly a hollow needle member. The needle shaft carrier is configured for facilitating insertion of a hollow needle shaft, and subsequent removal of the needle shaft guide member from the housing, prior to use of the hollow needle.

In another embodiment, the invention comprises a needle tip guard for placement on the shaft of a hollow needle, for positioning over a tip region of the hollow needle, following completion of use of the hollow needle, for isolating the tip region of the hollow needle, wherein a housing is operably configured to define a void region, and having a front end region and a rear end region, in which the housing has at least one passage therethrough operably configured to receive a shaft of a hollow needle therethrough. A shield member may be disposed within the void and configured for pivotable movement within the void between an aligned orientation permitting placement of a hollow needle shaft through the housing, and an unaligned orientation preventing passage of a hollow needle shaft through the front end region of the housing. An engagement member may be operably configured to be pivotable between a nonengagement position, when a hollow needle member is positioned in the housing extending from the front end region to the rear end region, and an engagement position, when a tip region of the hollow needle member has been withdrawn into the housing. The engagement member, in the nonengagement position permits the free movement of the hollow needle shaft relative to the housing, and in the engagement position exerting frictional force against the hollow needle shaft toward resisting movement of the hollow needle shaft relative to the housing. A biasing member, operably interengages the engagement member within the housing to prompt the engagement member toward the engagement position. An interconnecting member operably engages the engagement member with the shield member, and movable between a first position and a second position, wherein movement of the tip of the hollow needle shaft past the shield member, prompting the shield member to pivot, thus permitting the interconnecting member to move to a position permitting the engagement member to move to the engagement position.

A needle shaft carrier may be removably inserted into the housing, and extending from the front end region through to the rear end region thereof, the needle shaft carrier having a hollow configuration with an inner passage which is configured to insertingly a hollow needle member. The needle shaft carrier is preferably configured for facilitating insertion of a hollow needle shaft, and subsequent removal of the needle shaft guide member from the housing, prior to use of the hollow needle.

In yet another embodiment, the invention comprises a needle tip protector for placement on the shaft of a hollow needle, for positioning over a tip region of the hollow needle, following completion of use of the hollow needle, for isolating the tip region of the hollow needle, including a carrier including a substantially cylindrical tube having a hollow center, said cylindrical tube capable of being slidably mounted onto a needle. A needle guard is slidably mounted onto the carrier, such that the carrier and the needle guard may be simultaneously mounted onto the needle, but wherein the carrier may be removed from both the needle guard and the needle while leaving the needle guard slidably mounted on the needle. The needle guard preferably includes a locking shield which is prompted into a locking orientation covering the needle tip upon movement of the needle tip into the needle guard.

### BRIEF DESCRIPTION OF THE DRAWINGS

FIG. 1 is a front elevation of the needle tip protector according to one embodiment of the invention.

FIG. 2 is a side elevation of the needle tip protector shown in FIG. 1.

FIG. 3 is a front elevation of the needle tip protector shown in FIG. 1 as mounted onto the base of a needle.

FIG. 4 is a front elevation in cross-section of the needle tip protector shown in FIG. 1 as mounted onto a portion of a needle, with the shield in a loaded position.

FIG. 5 is a front elevation view in cross-section of the needle tip protector shown in FIG. 1, with the shield deployed into a locking orientation over the tip of the needle.

FIG. 6 is a top plan view of the needle tip protector shown in FIG. 1.

FIG. 7 is a bottom plan view of the needle tip protector shown in 1.

FIG. 8a is a side elevation, in section, of the shield shown in FIG. 4.

FIG. 8b is a bottom elevation of the shield shown in FIG. 4.

FIG. 8c is an end elevation of the shield shown in FIG. 4.

FIG. 9a is a side elevation, in section, of a shield according to an alternative embodiment of the invention.

FIG. 9b is a bottom elevation of the shield shown in FIG. 9a.

FIG. 9c is an end elevation of the shield shown in FIGS. 9a and 9b.

FIG. 10a is a side elevation of a housing for a needle guard according to an embodiment of the invention.

FIG. 10b is an end view of the needle guard housing according to the embodiment of FIG. 10a.

FIG. 10c is a view from the other end of the needle guard housing according to the embodiment of FIG. 10a.

FIG. 10d is a top view of the needle guard housing according to the embodiment of FIG. 10a.

FIG. 10e is a bottom view of the needle guard housing according to the embodiment of FIG. 10a.

FIG. 10f is a perspective view of an outer shell for use in combination with the needle guard housing of FIGS. 10a-10e.

FIG. 11a is a side view of a needle guard housing according to an alternative embodiment of the invention.

FIG. 11b is an end view of the needle guard housing according to the embodiment of FIG. 11a.

FIG. 11c is a bottom view of the needle guard housing according to the embodiment of FIG. 11a.

FIG. 11d is a top view of the needle guard housing according to the embodiment of FIG. 11a.

FIG. 12a is a schematic illustration of a needle tip guard, according to another alternative embodiment of the invention, shown with the needle in its “in use” position relative to the needle tip guard.

FIG. 12b is a schematic illustration of the needle tip guard according to the embodiment of FIG. 12a, showing the needle tip in its retracted position, with the guard engaged.

FIG. 13a is a schematic illustration of a needle tip guard, according to another alternative embodiment of the invention, shown with the needle in its “in use” position relative to the needle tip guard.

FIG. 13b is a schematic illustration of the needle tip guard according to the embodiment of FIG. 13a, showing the needle tip in its retracted position, with the guard engaged.

FIG. 14a is a plan view of a shield member, for use with the needle tip guard according to the embodiment of FIGS. 12a and 12b.

FIG. 14b is a side elevation of the shield member of FIG. 14a.

FIG. 15a is a plan view of a shield member, for use with the needle tip guard according to the embodiment of FIGS. 13a and 13b.

FIG. 15b is a side elevation of the shield member of FIG. 15a.

FIG. 16a is a side elevation of a needle tip guard according to an alternative embodiment of the invention.

FIG. 16b is a side elevation of the needle tip guard according to the embodiment of FIG. 16a, wherein the outer shell is omitted to show the shield member in its undeployed position.

FIG. 16c is a side elevation of the needle tip guard according to the embodiment of FIG. 16a, wherein the outer shell is omitted to show the shield member in its deployed position.

FIG. 16d is a side elevation, partially in section, of the needle tip guard according to the embodiment of FIG. 16a, wherein the interaction between the outer shell and the barbs of the housing is shown.

FIG. 16e is a view of the needle tip guard, shown turned 90 degrees from the view of FIG. 16a.

FIG. 17a is a side elevation of a needle tip guard according to an alternative embodiment of the invention.

FIG. 17b is a side elevation of the needle tip guard according to the embodiment of FIG. 17a, wherein the outer shell is omitted to show the shield member in its undeployed position.

FIG. 17c is a side elevation of the needle tip guard according to the embodiment of FIG. 17a, wherein the outer shell is omitted to show the shield member in its deployed position.

FIG. 17d is a side elevation, partially in section, of the needle tip guard according to the embodiment of FIG. 17a, wherein the interaction between the outer shell and the barbs of the housing is shown.

FIG. 17e is a view of the needle tip guard, shown turned 90 degrees from the view of FIG. 17a.

FIG. 18a is a side view of a needle tip guard according to another alternative embodiment of the invention, showing the guard with the outer shell omitted, to show an alternative shield member, in its undeployed position.

FIG. 18b is a side view of a needle guard according to the alternative embodiment of FIG. 18a, showing the guard with the outer shell omitted, to show the alternative shield member in its deployed position.

FIG. 19a is a side elevation, of a needle guard according to a variation of the embodiment of FIGS. 16a-16e, shown in its undeployed configuration.

FIG. 19b is a side elevation, of a needle guard according to a variation of the embodiment of FIGS. 16a-16e, shown in its deployed configuration.

FIG. 19c is a side elevation, of a needle guard according to a variation of the embodiment of FIGS. 17a-17e, shown in its undeployed configuration.

FIG. 19d is a side elevation, of a needle guard according to a variation of the embodiment of FIGS. 17a-17e, shown in its deployed configuration.

FIG. 20a is a side view of a needle guard according to another alternative embodiment of the invention, showing the guard in its undeployed position.

FIG. 20b is a side view of the needle guard of the embodiment of FIG. 20a, showing the guard in its deployed position.

FIG. 20c is a perspective view of the housing and moving components of the needle guard of FIGS. 20a and 20b.

FIG. 21a is a side view of a needle guard according to another alternative embodiment of the invention, having an operation similar to that of the embodiment of FIGS. 20a and 20b, and shown in its undeployed configuration.

FIG. 21b is a side elevation of the needle guard according to the embodiment of FIG. 21a, shown in its deployed configuration.

FIG. 21b is a perspective, partially exploded, view of the housing, block, and pivoting member of the guard of FIG. 21a.

FIG. 22a is a side view of a needle guard according to another alternative embodiment of the invention, having an operation similar to that of the embodiment of

FIGS. 21a and 21b, and shown in its undeployed configuration.

FIG. 22b is a side elevation of the needle guard of FIG. 22a, shown in its deployed configuration.

FIG. 22c is a perspective, partially exploded, view of the housing, block, and pivoting member of the guard of FIG. 22a.

FIG. 23a is a side elevation of another needle tip guard according to another alternative embodiment of the invention, showing the guard in its undeployed configuration.

FIG. 23b is a side elevation of the needle tip guard of FIG. 23a, showing it in its deployed configuration.

FIG. 23c is an exploded perspective view of the housing and principal moving components for the needle tip guard of FIGS. 23a and 23b.

### DETAILED DESCRIPTION OF THE INVENTION

While this invention is susceptible of embodiment in many different forms, there is shown in the drawings and will be described in detail several specific embodiments, with the understanding that the present disclosure is to be considered an exemplification of the principles of the invention and is not intended to limit the invention to the embodiments illustrated.

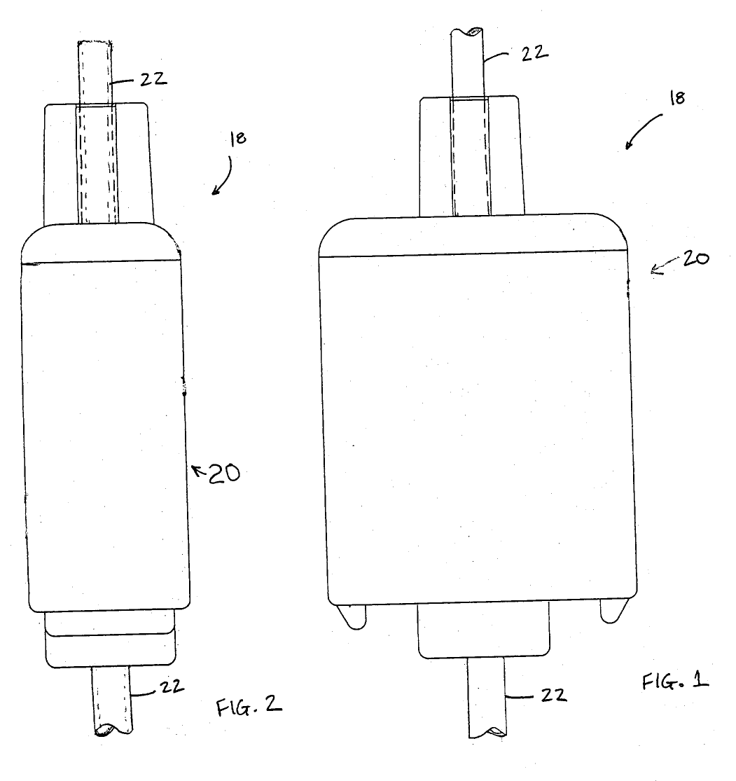

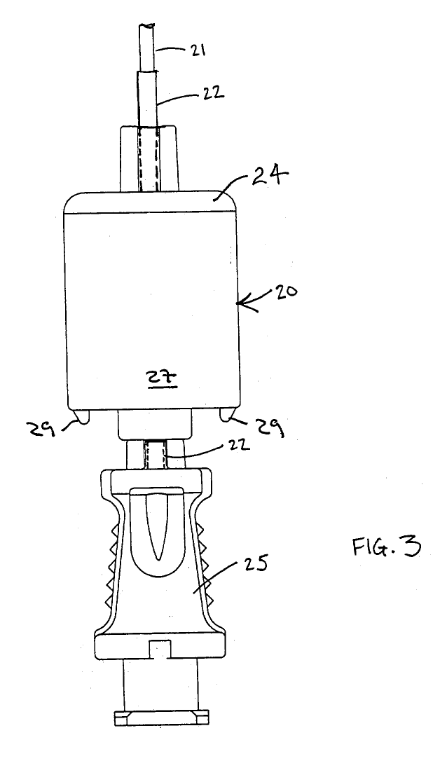

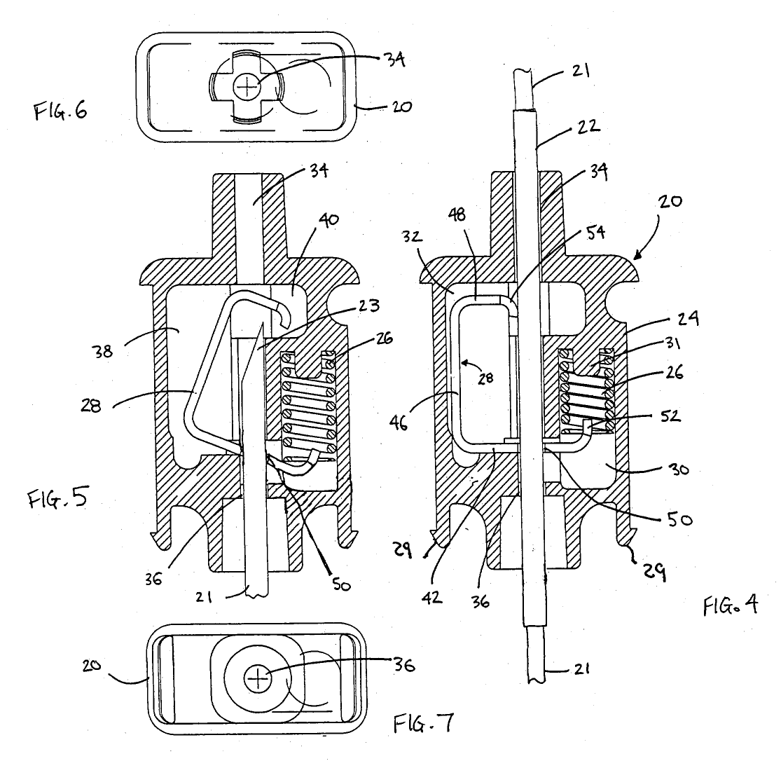

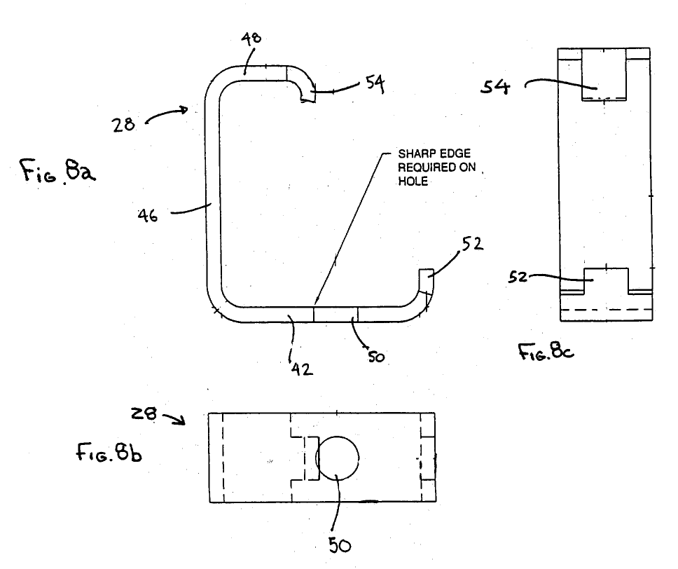

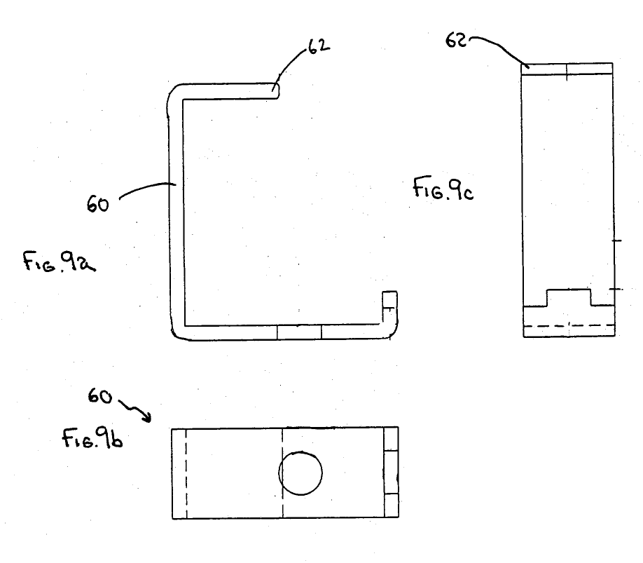

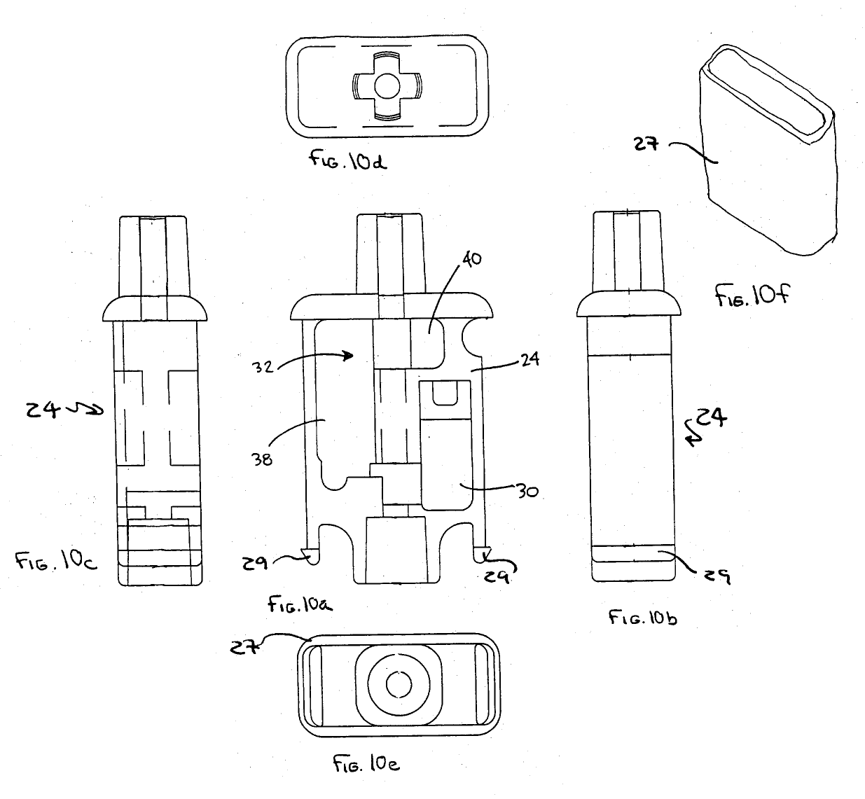

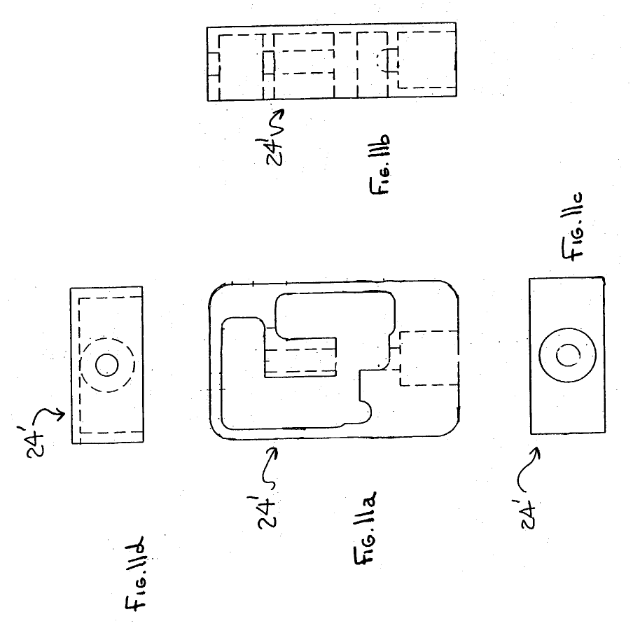

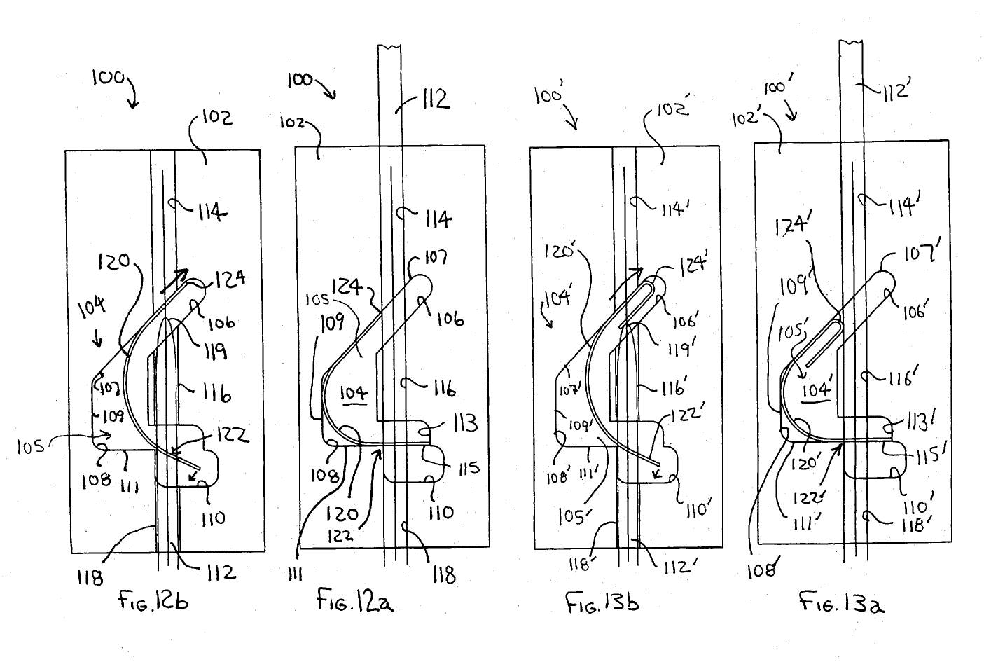

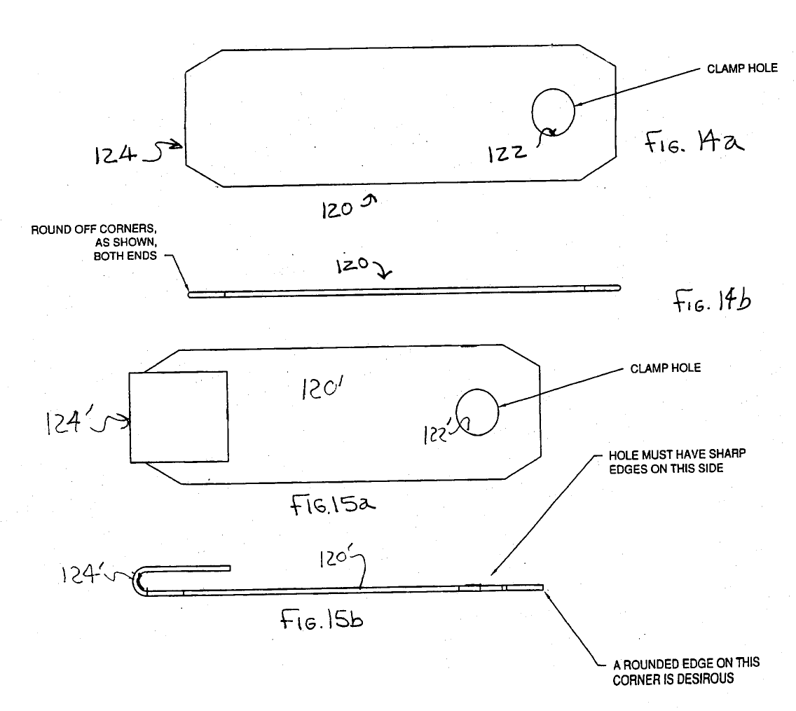

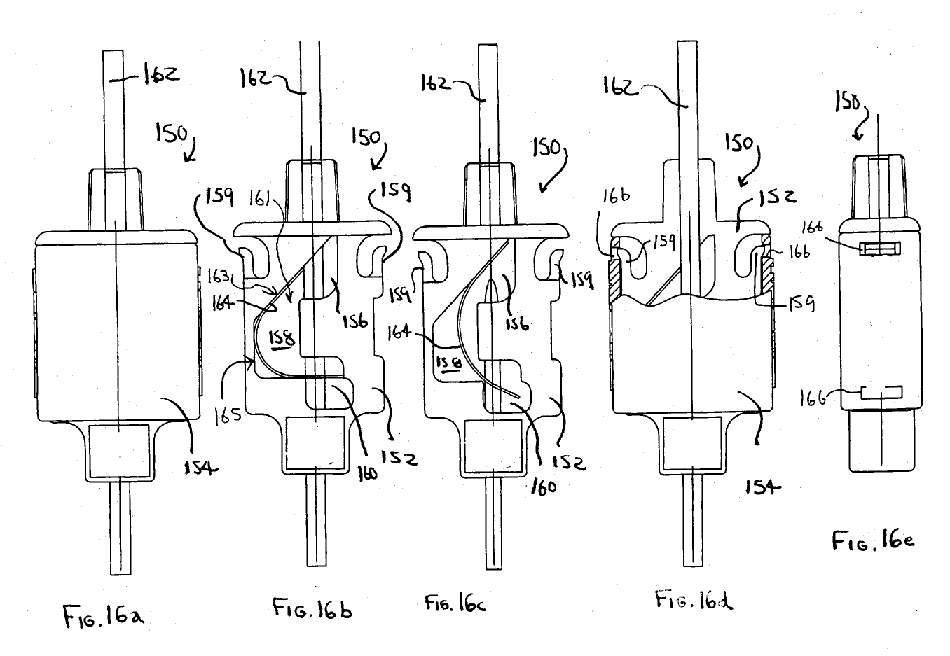

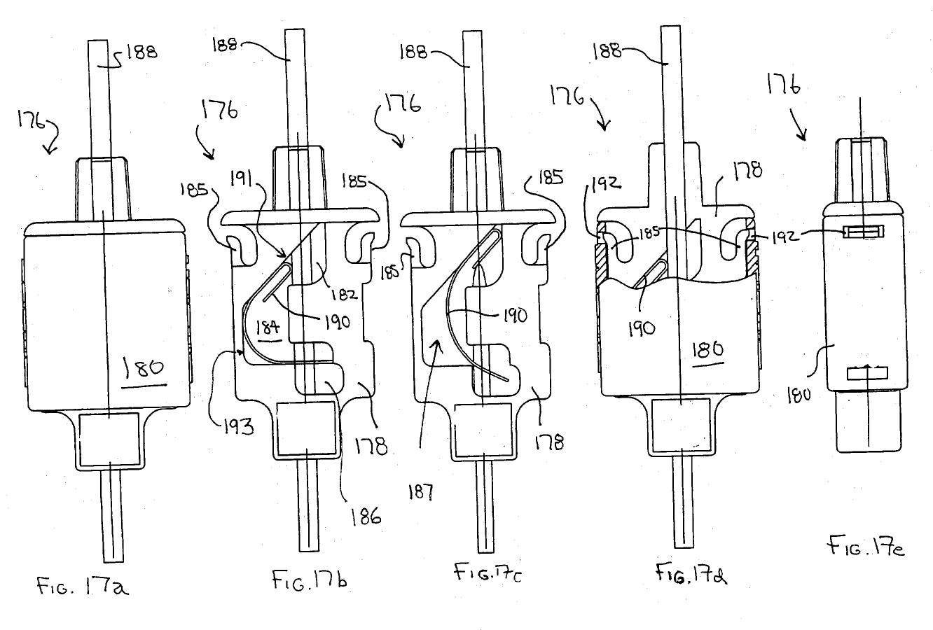

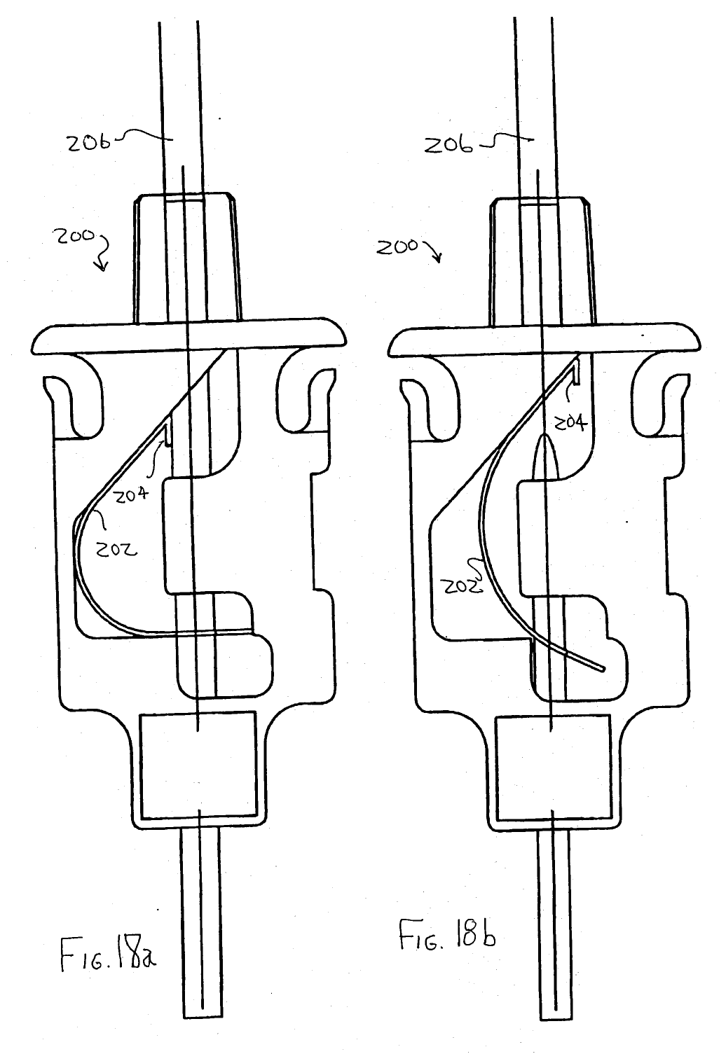

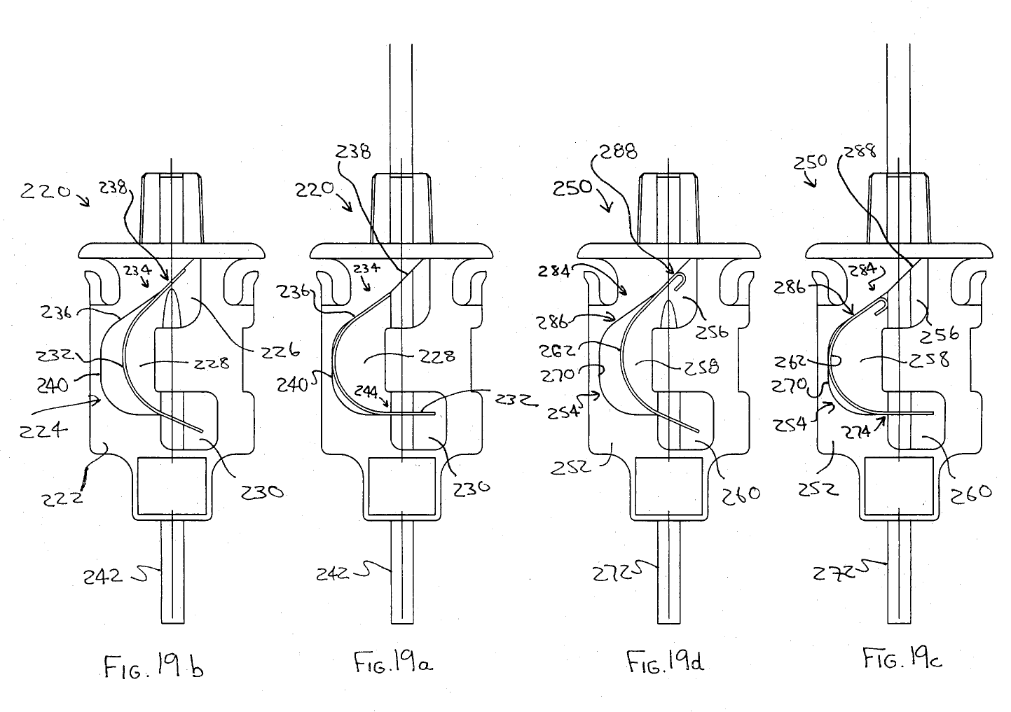

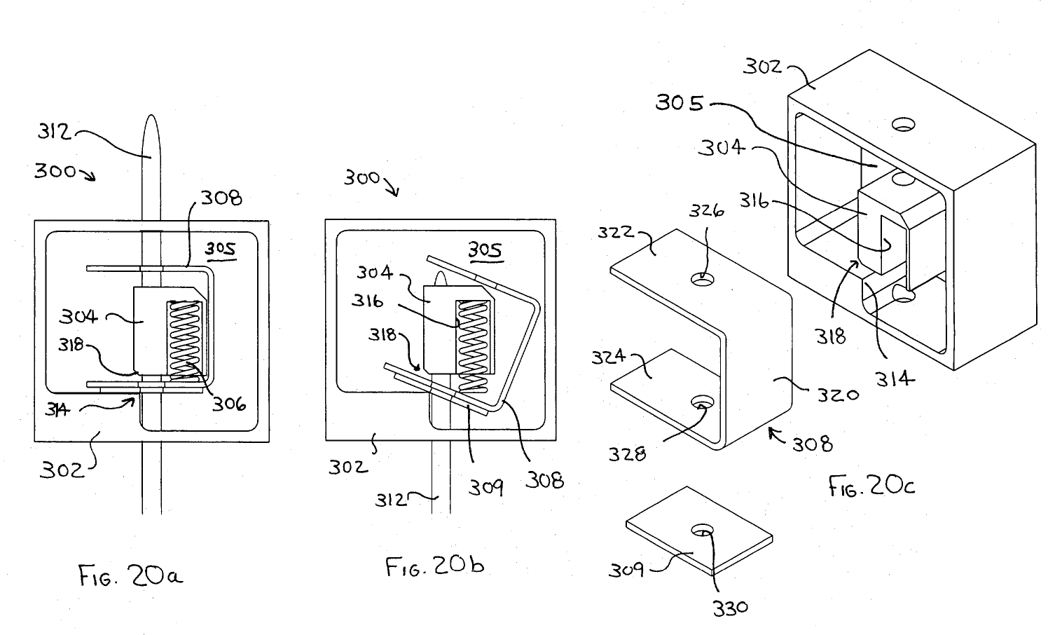

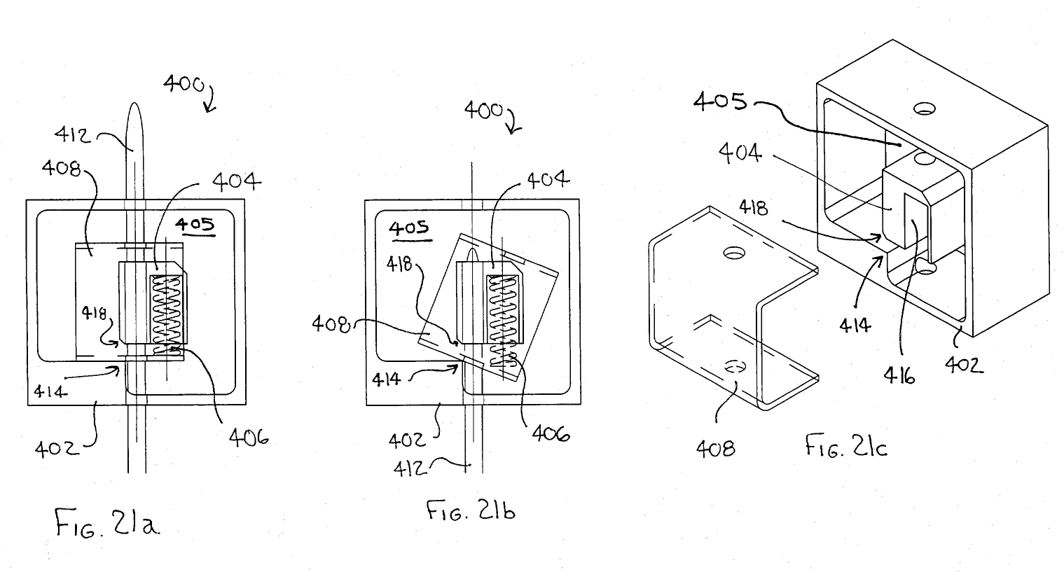

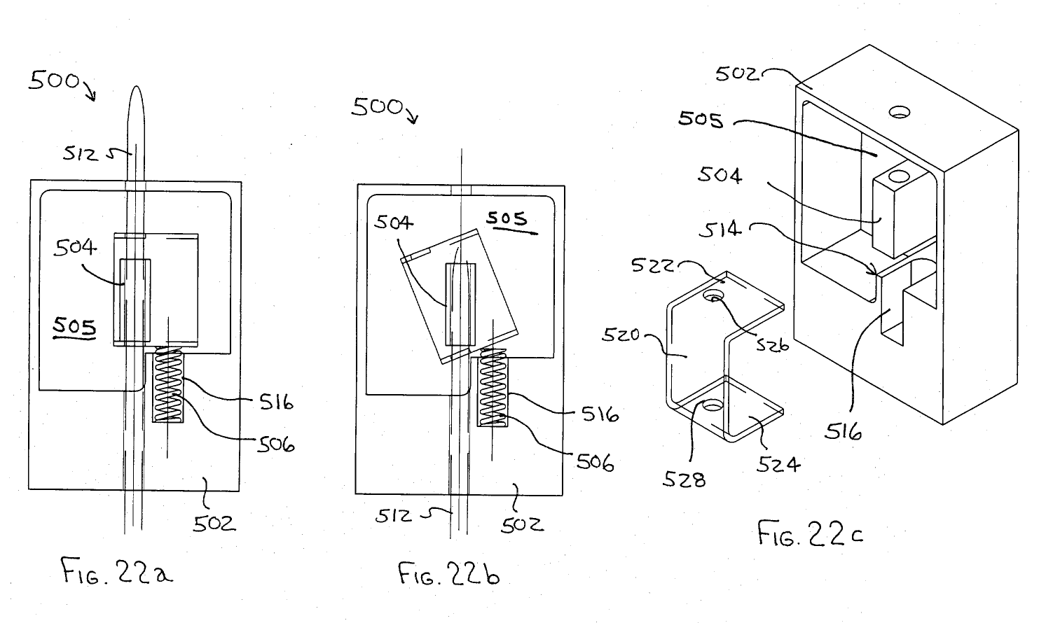

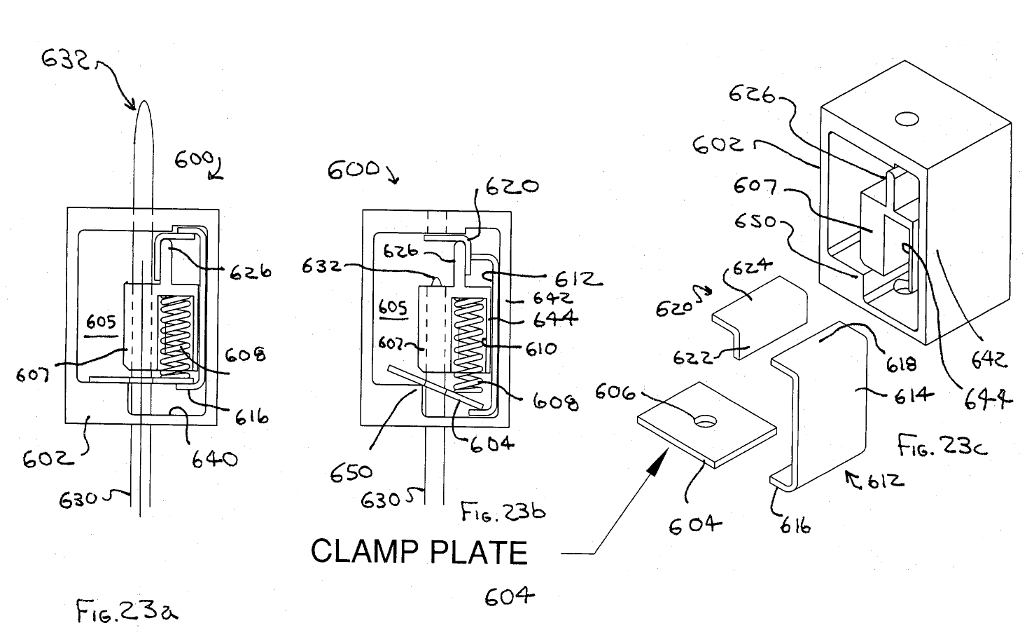

Needle tip guard 18 is shown in FIGS. 1-4 as including needle guard 20 and carrier (or loading tube) 22. At the outset, it must be noted that while needle tip guard 18 will be shown as used in combination with a standard hypodermic needle 21, it is contemplated that needle tip protector 18 may be used in combination with any number of medical needle-type devices. For instance, needle tip protector 18 may be used on hypodermic needles, biopsy needles, catheters, anesthesia needles, radiation needles mammography needles, etc. In essence, the needle tip protector is intended to be used in combination with any needle type device which includes a sharp tip which may be exposed after use of that needle type device. Further, while a specific needle tip protector is shown and described as used with a particular needle shape and diameter, it is contemplated that the needle tip protector may be used with various needles having any number of different shapes, diameters, lengths or tips.

Needle guard 20 is shown in FIGS. 1-7 as including housing 24, spring 26 and shield 28. Housing 24 is preferably constructed from a hard plastic material, while spring 26 and shield 28 are preferably constructed from a metallic material. However, it is certainly contemplated that alternative materials may be used in combination with the various components of the present invention. In addition, as shown in FIG. 3, an outer shell 27 is also provided, which is likewise preferably fabricated from a plastic material, although other materials may be employed, if desired. An analogous shell 27 is also shown in FIG. 10f. Moreover, while needle guide 20 is shown in the drawings as having a substantially rectangular parallelepiped configuration with rounded corners, with the exception of the very top and bottom portions of the needle guard, others shapes are likewise contemplated. For instance, the needle guard may comprise a substantially oval shape, or substantially cylindrical shape with a relatively circular diameter. Any number of shapes would work well when used in combination with the present invention, as would be known by those with ordinary skill in the art having the present disclosure before them.

In an embodiment of the invention, housing 24 may be provided with resiliently disposed barbs 29. Outer shell 27 may be a simple tube, having a rectangular cross-section (see, e.g., shell 27′ of FIG. 10f) which, once the internal components of guard 20 have been placed in housing 24, can be slid onto and over housing 24. Once barbs 29 have passed the edge of outer shell 29, as seen in FIG. 3, they spring outwardly, and prevent outer shell 29 from being readily removed from housing 24.

Making reference as well to FIGS. 4-7, housing 24 includes spring cavity 30, shield cavity 32, top needle aperture 34 and bottom needle aperture 36. Spring cavity 30 is preferably dimensioned to house spring 26, and includes a seat 31 in which spring is retained. Shield cavity 32 is preferably a substantially inverted “L” shaped opening in housing 24, taking an inverted L-shaped configuration as compared to L-shaped shield 28. In particular, shield cavity 32 includes resting chamber 38, namely an elongated vertical chamber, and a locking chamber 40, which is designed to accept shield 28 in its locking position when needle guard 20 is moved past needle tip 23. Shield cavity 32 allows spring 26 to cantilever shield 28 into the locking orientation when the needle tip is pulled into needle guard 20.

Top needle aperture 34 and a bottom needle aperture 36 are formed in the top and bottom extension portions of needle guard 20. Top needle aperture 34 and bottom needle aperture 36 are preferably aligned to accept cylindrically shaped carrier 22, and to eventually, in turn, accept needle 21. As will be described in more detail below, carrier 22 may be removed upon mounting of needle tip protector 18 to needle 21, thus allowing positioning the needle shaft in top needle aperture 34 and bottom needle aperture 36. The top and bottom apertures 34, 36, are preferably only slightly larger in diameter than the carrier and needle, thus allowing a stable sliding movement of needle guard 20 back and forth along the needle shaft.

Shield 28 preferably includes bottom horizontal leg 42, vertical leg 46 and upper horizontal leg 48. Bottom horizontal leg 42 includes aperture 50 and spring contacting lip 52. The diameter of aperture 50 is preferably only slightly larger than the diameter of carrier 22, which is slightly larger than diameter of needle 21, to help maintain shield 28 in a proper loaded, non-locking orientation, shown in FIG. 4. Aperture 50 in shield 28 also acts in combination with top needle aperture 34 and bottom needle aperture 36 in housing 24 to facilitate slidable movement of needle guard 20 on the needle shaft. Spring contacting lip 52, shown in FIGS. 4, 5, and 8, preferably contacts the bottom portion of spring 26, such that spring 26 exerts a force on spring contacting lip 52, and thus shield 28. Bottom horizontal leg 42 is preferably in contact with housing 24, specifically with the bottom portion of resting chamber 38.

Vertical leg 46 spans the distance between bottom horizontal leg 42 and upper horizontal leg 48. As can be seen in FIG. 4, a portion of vertical leg 46 preferably contacts the inner portion of housing 24. However, it is likewise contemplated that no such contact is necessary.

Upper horizontal leg 48 includes needle riding lip 54. As can be seen from FIG. 4, needle riding lip initially contacts carrier 22, and then eventually needle 21 after carrier 22 is removed, as needle tip protector 18 is moved back and forth along the needle. The needle riding lip extends downwardly to provide the needle with a relatively flat bracing surface, which helps hold shield 28 into place before the needle withdrawn into needle guard 20 and shield 28 is deployed.

Alternatively, and as is shown in FIGS. 9a-9c, an alternative shield 60 may employed, including upper horizontal leg 62 having no vertically extending lip. Accordingly, the end face of upper horizontal leg 62 would ride directly against carrier 22, and then against needle 21 after removal of the carrier.

Carrier 22 is shown in FIGS. 1-4 as comprising a substantially hollow, cylindrical tube on which needle guard 20 is mounted, or more alternatively considered, carrier 22 has been inserted into needle guard 20, prior to insertion of a needle. In particular, carrier 22 extends through top needle aperture 34, bottom needle aperture 36, as well as aperture 50 in shield 28, and is slidably disposed relative thereto. Carrier 22 extends beyond the top and bottom portions of needle guard 20 so that a user may grasp either end of carrier 22. The exact distance by which carrier 22 extends beyond the top and bottom portions of needle guard 20 may be varied, depending upon the particular application, user preference, etc. It is preferred, however, that carrier extends far enough on both sides of needle guard 20 such that a user can grasp carrier 22 to remove carrier from both needle guard 20, as well as needle 21.

Further, it is preferred that carrier 22 slides relatively freely within needle guard 20, as well on needle 21. As is the case with needle 21 when carrier 22 is removed from needle guard 20 and taken off of needle 21, shield portion 28 of needle guard 20 helps maintain carrier 22 in a firm, friction-based contact with needle guard 20. Further, it is preferred that the needle guard includes dulled or otherwise non-sharpened ends. This allows the advantageous placement of needle guard 20 onto carrier 22 without the risk of incurring an inadvertent carrier puncture wound during the mounting process, and especially without damaging the sharpened tip of the needle, which may otherwise occur, if an attempt is made to insert a needle through guard 20, in the absence of carrier 22.

In operation, and is best seen in FIGS. 1-5, needle tip protector 18 including needle guard 20 mounted on carrier 22 is initially mounted on needle 21. As is shown in FIG. 3, needle tip protector 18 is preferably positioned proximate to or toward base portion 25 of needle 21. This allows a user clear vision and access to the tip of the needle, and minimizes the intrusiveness of needle tip protector 18 in any given medical procedure, whether it is a simple shot, a biopsy or a mammography procedure. Notably, carrier 22 is preferably slightly larger than needle 21, such that there is little or no frictional contact between carrier 22 and needle 21, to enable needle 21 to be inserted with ease. Once the needle 21 has been passed through guard 20, then carrier 22 is promptly removed. There will be frictional contact between needle 21 and the internal workings of guard 20, to preserve needle guard 20 in place once mounted onto needle 21. Thus, it is preferred that needle guard 20 is slidably moveable along the needle shaft, but also frictionally held in place once positioned in a desired location on the needle shaft.

Before the needle is used for its desired purpose, carrier 22 is preferably removed from needle tip protector 18 to leave needle guard 20 mounted by itself on needle 21. This removal is preferably done soon after insertion of needle 21, although it could be put off. This can be done by simply pulling carrier 22 off of needle 21, or by using a particular removal tool, such as a pair of pliers, which does not damage the needle. When carrier 22 is slid past shield 28, aperture 50 in the bottom part of shield 28 surrounds needle 21, while needle riding lip 54 in upper horizontal leg 48 of shield 28 rests up against needle 21. Thus, needle guard 20 becomes slidably movable along the needle shaft. The carrier may be easily disposed of.

After the needle has been used for its intended purpose, the needle guard 20 is moved along the needle shaft toward needle tip 23. Just as needle tip 23 slides past and beyond needle riding lip 54 of shield 28, spring 26 prompts shield 28 to move in a cantilevering fashion. In particular, spring 26 rocks shield 28 forward, thus forcing upper horizontal leg 48 of shield 28 into locking chamber 40 of shield cavity 32. As can be seen from FIG. 5, the upper portion of shield covers needle tip 23, thus preventing the needle from being moved back through the top of needle guard 20, and into a position where it may harm an individual, or be reused.

At the same time, the sides of aperture 50 of bottom horizontal leg 42 of shield 28 act to grab and freeze needle 21 relative to needle guard 20. In particular, the cantilevering motion of shield 28 forces the sides of aperture 50 to at least partially dig into needle 21, thus preventing needle 21 from sliding further away from and out of needle guard 20, through the bottom side of needle guard 20. Thus, needle guard 20 is locked directly to needle 21, with spring 26 exerting the cantilevering force on shield 28 to maintain the needle protector or guard in a position over needle tip 23. This prevents reuse of needle and prevents inadvertent punctures or injuries which may result from individuals brushing up against the sharp needle tip during disposable or after use.

FIGS. 10a-10e illustrate housing 24 which is substantially identical to shell 24 of FIGS. 1-5 (and accordingly due to the similarity of structure, like reference numerals are employed for the views of FIGS. 1-7 and FIGS. 10a-10e), as well as shell 27, which can be a simple tube of substantially rectangular cross-section, with rounded corners.

FIG. 11a is a side view of a needle guard housing 24′ according to an alternative embodiment of the invention, wherein the barbs for affixing the housing to an outer shell (not shown) are omitted. FIG. 11b is an end view of the needle guard housing 24′ according to the embodiment of FIG. 11a. FIG. 11c is a bottom view of the needle guard housing 24′ according to the embodiment of FIG. 11a. FIG. 11d is a top view of the needle guard housing 24′ according to the embodiment of FIG. 11a.

FIG. 12a is a schematic illustration of a needle tip guard (minus the carrier or loading tube), according to another alternative embodiment of the invention, shown with the needle in its “in use” position relative to the undeployed needle tip guard. While the carrier or loading tube is omitted from the drawings of this embodiment, it is understood that the guard of this embodiment may be initially provided with a cylindrical tubular carrier, in a manner substantially identical to that disclosed in the embodiment(s) of FIGS. 1-3. Needle tip guard 100 is provided with a housing, generally referred to by reference numeral 102, in which is positioned a void 104, which may be generally considered as comprising contiguously arranged chambers 106, 108 and 110, with bearing walls 107, 109 and 111. The outer shell has been omitted from the drawings for clarity of the illustrations. However, it is to be understood that a shell, such as that shown and described with respect to FIGS. 1-10f, may be employed. At the back of void 104 is located back wall 105. In a preferred embodiment wall 107 deviates 45 degrees from wall 109, which, in turn, is preferably perpendicular to wall 111, although some deviations from these respective angular relationships may be employed, if desired. As with the previously described embodiments, while housing 102 may have a generally rectangular parallelepiped configuration, any other suitable configuration may be used. The walls of chambers 106, 108 and 110 may be arranged to extend generally perpendicular to the plane of FIG. 12a. Needle shaft 112 extends longitudinally through housing 102, in particular through coaxially aligned cylindrical passages 114, 116 and 118.

Shield member 120, in the embodiment of FIGS. 12a and 12b, comprises a thin flat sheet of metal, e.g., stainless steel, as shown in FIGS. 14a and 14b. The material may be varied, so long as it has sufficient flexibility and resiliency, to withstand being bent, as shown in FIGS. 12a and 12b, without permanently deforming or suffering a significant loss of spring force (for extended shelf life). Shield member 120 is provided with aperture 122, through which needle shaft 112 is inserted (FIGS. 12a and 12b). Preferably, aperture 122 is either oval or is circular with a diameter which is somewhat greater than the outside diameter of needle shaft 112.

When needle shaft 112 is in its “in use” position, with respect to needle guard 100, shield member 120 is substantially bent, such that the respective ends of shield member 120 have an acute angle between them, of 45 degrees, more or less, and edge 124 of shield member 120 is being pushed (by the resilient spring force stored in member 120) against needle shaft 112. Wall 113 of housing 102 is provided, to act as a locator, giving shield member 120 a surface to butt up against, serving to center aperture 122, relative to the center of needle shaft 112.

When the use of the needle has been completed, needle guard 100 is pushed toward and past the needle tip 119 (FIG. 12b). As soon as needle tip 119 passes edge 124 of shield member 120, the stored spring force in shield member 120 causes it to be prompted to “straighten out”. Edge 124 will tend to move up and to the right as shown in FIG. 12b, while the opposite end of shield member 120 will tend to rotate clockwise, until the edges of aperture 122 come into contact with the surface of needle shaft 112. Thus, the upper end of shield member 120 prevents needle tip 119 from being pushed out of needle guard 100, while the lower end of shield member 120, specifically the upper and lower edges of aperture 122 frictionally engage the sides of needle shaft 112, thus providing resistance to the removal of needle shaft 112, through the bottom of needle guard 100.

FIG. 13a is a schematic illustration of a needle tip guard, according to another alternative embodiment of the invention, shown with the needle in its “in use” position relative to the needle tip guard. FIG. 13b is a schematic illustration of the needle tip guard according to the embodiment of FIG. 13a, showing the needle tip in its retracted position, with the guard engaged. While the carrier or loading tube is omitted from the drawings of this embodiment, it is understood that the guard of this embodiment may be initially provided with a cylindrical tubular carrier, in a manner substantially identical to that disclosed in the embodiment(s) of FIGS. 1-3.

To the extent that the structures of FIGS. 13a, 13b, 15a, 15b are similar to, and have similar functions, to like elements in the embodiment of FIGS. 12a, 12b, 14a, 14b, like reference numerals, augmented by a prime (′) will be employed.

Needle tip guard 100′ is provided with a housing, generally referred to by reference numeral 102′, in which is positioned a void 104′, which may be generally considered as comprising contiguously arranged chambers 106′, 108′ and 110′, with bearing walls 107′, 109′ and 111′. At the back of void 104′ is back wall 105′. The outer shell has been omitted from the drawings for clarity of the illustrations. However, it is to be understood that a shell, such as that shown and described with respect to FIGS. 1-10f, may be employed. In a preferred embodiment wall 107′ deviates 45 degrees from wall 109′, which, in turn, is preferably perpendicular to wall 111′, although some deviations from these respective angular relationships may be employed, if desired. As with the previously described embodiments, while housing 102′ may have a generally rectangular parallelepiped configuration, any other suitable configuration may be used. The walls of chambers 106′, 108′ and 110′ may be arranged to extend generally perpendicular to the plane of FIG. 12a. Needle shaft 112′ extends longitudinally through housing 102′, in particular through coaxially aligned cylindrical passages 114′, 116′ and 118′. Wall 113′ of housing 102′ is provided, to act as a locator, serving to center aperture 122′, relative to the center of needle shaft 112′. While wall 113′ does provide some frictional resistance to the end 115′ of shield member 120′, when guard 100′ is deployed (as described in further detail hereinafter), the resistance is not so much that it cannot be overcome upon deployment of guard 100′.

Shield member 120′, in the embodiment of FIGS. 13a and 13b, comprises a thin, generally flat, sheet of metal, e.g., stainless steel, as shown in FIGS. 15a and 15b, except that instead of presenting a simple straight edge at its upper end, as in the embodiment of FIGS. 12a, 12b, and 14a and 14b, the upper end of shield member 120′ has been folded back upon itself and curled under to form an elongated U-shaped portion, to present a rounded end 124′. The material may be varied, so long as it has sufficient flexibility and resiliency, to withstand being bent, as shown in FIGS. 13a and 13b, without permanently deforming or suffering a significant loss of spring force (for extended shelf life). Shield member 120′ is provided with aperture 122′, through which needle shaft 112′ is inserted (FIGS. 13a and 13b). Preferably, aperture 122′ is either oval or is circular with a diameter which is somewhat greater than the outside diameter of needle shaft 112′.

When needle shaft 112′ is in its “in use” position, with respect to needle guard 100′, shield member 120′ is substantially bent, such that the respective ends of shield member 120′ have an acute angle between them, of 45 degrees, more or less, and rounded end 124′ of shield member 120′ is being pushed (by the resilient spring force stored in member 120′) against needle shaft 112′.

When the use of the needle has been completed, needle guard 100′ is pushed toward and past the needle tip 119′ (FIG. 13b). As soon as needle tip 119′ passes rounded end 124′ of shield member 120′, the stored spring force in shield member 120′ causes it to be prompted to “straighten out”. Rounded end 124′ will tend to move up and to the right as shown in FIG. 13b, while the opposite end of shield member 120′ will tend to rotate clockwise, until the edges of aperture 122′ come into contact with the surface of needle shaft 112′. Thus, the upper end of shield member 120′ prevents needle tip 119′ from being pushed out of needle guard 100′, while the lower end of shield member 120′, specifically the upper and lower edges of aperture 122′ frictionally engage the sides of needle shaft 112′, thus providing resistance to the removal of needle shaft 112′, through the bottom of needle guard 100′.

FIGS. 16a-16e illustrate a needle tip guard 150, which has a configuration which is substantially similar to needle tip guard 100 of FIGS. 12a, 12b, 14a and 14b, except that the manner of engagement between housing 152 and shell 154 is somewhat different. Guard 150 employs a flat shield member 164. Housing 152 has chambers 156, 158 and 160 therein, backed by back wall 161, which interact with needle shaft 162, and flat shield member 164, in a manner substantially identical to that of the embodiment of FIGS. 12a, 12b, 14a and 14b. Accordingly, the operation of those components is understood to be the same and no further detailed discussion is required. While the carrier or loading tube is omitted from the drawings of this embodiment, it is understood that the guard of this embodiment may be initially provided with a cylindrical tubular carrier, in a manner substantially identical to that disclosed in the embodiment(s) of FIGS. 1-3. Chambers 156 and 158 are provided with walls 163 and 165 respectively, which diverge from one another at a 45 degree angle, as in the embodiment of FIGS. 12a-b and 13a-b.

Instead of barbs 29, being positioned at an end of the housing, pointing away from the needle tip, as in the embodiment of FIGS. 1-10f, in the embodiment of FIGS. 16a-16e (in which needle shaft 162 is omitted), barbs 159 are positioned near the end of housing 152, “pointing” toward the needle tip. Shell 154 is provided with slots 166, in its side walls, which may be located near each end. During assembly, when shell 154 is initially being pushed onto housing 152, the tips of barbs 159 must be pushed inwardly, toward the interior of housing 152. As shell 154 is pushed over housing 152, once the tips of barbs 159 encounter slots 166, the stored spring force in barbs 159 causes them to spring outwardly and enter slots 166, thus securing housing 152 within shell 154.

FIGS. 17a-17e illustrate a needle tip guard 176, which has a configuration which is substantially similar to needle tip guard 150 of FIGS. 16a-16e, except that the shield member is like that of the embodiment of FIGS. 13a, 13b, 15a and 15b, namely having a U-shaped upper end. Otherwise, the manner of engagement between housing 178 and shell 180 is the same as in the embodiment of FIGS. 16a 163. While the carrier or loading tube is omitted from the drawings of this embodiment, it is understood that the guard of this embodiment may be initially provided with a cylindrical tubular carrier, in a manner substantially identical to that disclosed in the embodiment(s) of FIGS. 1-3. Guard 176 employs a shield member 190 having a curved under U-shaped end. Housing 178 has chambers 182, 184, 186 therein, backed by back wall 187, which interact with needle shaft 188, and shield member 190, in a manner substantially identical to that of the embodiment of FIGS. 13a, 13b, 15a and 15b. Accordingly, the operation of those components is understood to be the same and no further detailed discussion is required. Chambers 182 and 184 include walls 191 and 193, respectively, that diverge at a 45 degree angle, preferably, as in the embodiment of FIGS. 12a-b and 13a-b.

As in the embodiment of FIGS. 16a-16e (in which needle shaft 162 is omitted), barbs 185 are positioned near the end of housing 178, “pointing” toward the needle tip. Shell 180 is provided with slots 192, in its side walls, which slots may be located near each end edge of shell 180. During assembly, when shell 180 is initially being pushed onto housing 178, the tips of barbs 185 must be pushed inwardly, toward the interior of housing 178. As shell 180 is pushed over housing 178, once the tips of barbs 185 encounter slots 192, the stored spring force in barbs 185 causes them to spring outwardly and enter slots 192, thus securing housing 178 within shell 180.

FIG. 18a is a side view of a needle tip guard 200 according to another alternative embodiment of the invention, showing the guard with the outer shell omitted, to show an alternative shield member 202, in its undeployed position. While the carrier or loading tube is omitted from the drawings of this embodiment, it is understood that the guard of this embodiment may be initially provided with a cylindrical tubular carrier, in a manner substantially identical to that disclosed in the embodiment(s) of FIGS. 1-3. Guard 200 operates in a manner substantially similar to that of the guards of the embodiments of FIGS. 16a-16e and 17a-17e, except that in this embodiment, shield member 202 has a downwardly (i.e., away from the direction of the needle tip) projecting guide lip 204. In its undeployed position, the guide lip 204 of shield member 202 bears against the side surface of needle shaft 206.

FIG. 18b is a side view of a needle guard according to the alternative embodiment of FIG. 18a, showing the guard with the outer shell omitted, to show the alternative shield member in its deployed position.

FIGS. 19a-19b illustrate a needle tip guard 220, which has a configuration which is substantially similar to needle tip guard 150 of FIGS. 16a-16e. Guard 220 includes housing 222, with interior void 224, forming chambers 226, 228 and 230. Guard 220 further includes shield member 232, which may be substantially identical to shield member 120 of FIGS. 14a-14b. However, unlike the embodiments of FIGS. 12a-18b, in this embodiment, the wall 234 against which shield member 232 slides is not straight; rather, it has a shallow angled portion 236, which proceeds at an angle of, for example, 35 degrees from the “horizontal” as FIG. 19b is viewed (that is, at, preferably, 55 degrees divergence to wall 240), whereas portion 238 is steeper, extending at a 45 degree angle to the horizontal and to wall 240. Also, wall 240 is preferably proportionally longer than the similarly situated walls in the other embodiments. This combination permits shield member 232 to be bent with a larger relative radius of curvature, as compared to the previously described embodiments. This makes installation of the shield member 232 into housing 222 easier, and puts less stress on shield member 232, in its undeployed position, thus reducing the likelihood of fatigue failure, awaiting use. In addition, the embodiment of FIGS. 19a, 19b lacks a wall in chamber 230 that is analogous to wall 113 of FIGS. 12a, 12b. Removal of the wall is seen to make the tooling for forming housing 222 simpler and less expensive, and may alleviate possible issues of binding of the end of the shield against that wall, that may occur in the embodiment of FIGS. 12a-b, for example. Accordingly, as seen in FIG. 19a, in its undeployed configuration, the lower end of shield member 232 may not be at a substantially perpendicular angle to needle shaft 242, as in the other previously discussed embodiments. Also, because the centering function is not provided, an edge of the aperture 244 at the lower end may be bearing slightly against the side of needle shaft 242. However, this frictional force will not be sufficient to prevent movement of shaft 242 relative to housing 222, towards deployment of guard 220. However, once deployment has occurred, the lower end of shield member 232 adopts an oblique angle with respect to needle shaft 242, with substantially increased frictional force acting to resist further movement of needle shaft 242, relative to housing 222. While the carrier or loading tube is omitted from the drawings of this embodiment, it is understood that the guard of this embodiment may be initially provided with a cylindrical tubular carrier, in a manner substantially identical to that disclosed in the embodiment(s) of FIGS. 1-3.

FIGS. 19c-19d illustrate a needle tip guard 250, which has a configuration which is substantially similar to needle tip guard 176 of FIGS. 17a-17e. Guard 250 includes housing 252, with interior void 254, forming chambers 256, 258 and 260. Guard 250 further includes shield member 262, which may be substantially identical to shield member 120′ of FIGS. 15a-15b. However, unlike the embodiments of FIGS. 12a-18b, in this embodiment, the wall 284 against which shield member 252 slides is not straight; rather, it has a shallow angled portion 286, which proceeds at an angle of, e.g., 35 degrees from the “horizontal” as FIG. 19c is viewed (that is, at, preferably, 55 degrees divergence to wall 240), whereas portion 288 is steeper, extending at a 45 degree angle to the horizontal and to wall 270. Also, wall 270 is preferably proportionally longer than the similarly situated walls in the other embodiments. This combination permits shield member 262 to be bent with a larger relative radius of curvature, as compared to the previously described embodiments. This makes installation of the shield member 262 into housing 252 easier, and puts less stress on shield member 252, in its undeployed position, thus reducing the likelihood of fatigue failure, awaiting use. In addition, the embodiment of FIGS. 19c, 19d lacks a wall in chamber 260 that is analogous to wall 113 of FIGS. 12a, 12b. Removal of the wall is seen to make the tooling for forming housing 252 simpler and less expensive. Accordingly, as seen in FIG. 19c, in its undeployed configuration, the lower end of shield member 262 may not be at a substantially perpendicular angle to needle shaft 272, as in the other previously discussed embodiments. Also, because the centering function is not provided, an edge of the aperture 274 at the lower end may be bearing slightly against the side of needle shaft 272. However, this frictional force will not be sufficient to prevent movement of shaft 272 relative to housing 252, towards deployment of guard 250. However, once deployment has occurred, the lower end of shield member 262 adopts an oblique angle with respect to needle shaft 272, with substantially increased frictional force acting to resist further movement of needle shaft 272, relative to housing 252. While the carrier or loading tube is omitted from the drawings of this embodiment, it is understood that the guard of this embodiment may be initially provided with a cylindrical tubular carrier, in a manner substantially identical to that disclosed in the embodiment(s) of FIGS. 1-3.

A still further alternative embodiment of the invention is shown in FIGS. 20a, 20b and 20c. While the carrier or loading tube is omitted from the drawings of this embodiment, it is understood that the guard of this embodiment may be initially provided with a cylindrical tubular carrier, in a manner substantially identical to that disclosed in the embodiment(s) of FIGS. 1-3. In this embodiment, needle tip guard 300 includes a housing 302 with suitable bores for passage therethrough of a needle shaft, an inner support block 304, biasing member (i.e., spring) 306, pivoting member 308 and clamp plate 309. Inner support block 304 emanates from back wall 305 of housing 302. Housing 302 is surrounded by an outer shell which may have any desired configuration (such as a hollow rectangular tube, as shown and described with respect to previously shown embodiments), and thus which has been omitted for clarity of the illustration of the inner components. Housing 302 is configured to slidably move along needle shaft 312, and may have any desired configuration, so long as it provides an interior corner 314 to act as a fulcrum for pivoting member 308, as described hereinafter. Block 304 is provided with a through bore, to permit the free sliding movement of needle shaft 312 therethrough. Block 304 also has a blind bore 316, for receiving biasing member 306, and a diagonal corner portion 318, which is provided to give room for the pivoting member 308. Pivoting member 308 has a web 320, which connects arms 322, 324. Arm 322 has an aperture 326, which is configured to let needle shaft 312 pass freely therethrough. Arm 324 is provided with aperture 328 which may be in the form of an elongated ellipse, or of sufficiently large diameter, so as not to bind on needle shaft 312, when pivoting member 308 is in the deployed configuration of FIG. 20b. Beneath pivoting member 308, between arm 324 and the interior corner 314 is positioned clamp plate 309, which has an aperture 330, which is slightly larger than the diameter of needle shaft 312, and which further has sharp corners therein, to grip into needle shaft 312, when pivoting member 308 is in its deployed position.

When needle guard 300 is in the position of FIG. 20a, biasing member 306 is in compression. When guard 300 is slid down needle shaft 312, as soon as the needle tip clears aperture 326, the lateral force exerted by needle shaft 312 against the interior surface of aperture 326 (in reaction to the force being exerted by biasing member 306 to pivot pivoting member 308) is removed, and pivoting member 308 is free to pivot, around corner 314, to the extent that diagonal portion 318 permits. During this pivoting the inside corner edges of aperture 330 of clamp plate 309 will engage needle shaft 312, providing frictional resistance to the further sliding of needle shaft 312, relative to guard 300. Arm 322 will have sufficient length to prevent the reemergence of the needle tip out of the front of guard 300.

A still further alternative embodiment of the invention is shown in FIGS. 21a, 21b and 21c. While the carrier or loading tube is omitted from the drawings of this embodiment, it is understood that the guard of this embodiment may be initially provided with a cylindrical tubular carrier, in a manner substantially identical to that disclosed in the embodiment(s) of FIGS. 1-3. In this embodiment, needle tip guard 400 includes a housing 402 with suitable bores for passage therethrough of a needle shaft, an inner support block 404, biasing member (i.e., spring) 406, and pivoting member 408. Housing 402 is provided with a back wall 405, from which block 404 projects. Housing 402 is surrounded by an outer shell which may have any desired configuration, and thus which has been omitted for clarity of the illustration of the inner components. Housing 402 is configured to slidably move along needle shaft 412, and may have any desired configuration, so long as it provides an interior corner 414 to act as a fulcrum for pivoting member 408, which is rotated 90 degrees with respect to block 404, relative to the spatial relationship between block 304 and pivoting member 308, of the embodiment of FIGS. 20a and 20b. Block 404 is provided with a through bore, to permit the free sliding movement of needle shaft 412 therethrough. Block 404 also has a blind bore 416, for receiving biasing member 406, and a diagonal corner portion 418, which is provided to give room for the pivoting member 408. Pivoting member 408 has a web 420, which connects arms 422, 424. Arm 422 has an aperture 426, which is configured to let needle shaft 412 pass freely therethrough. Arm 424 is provided with aperture 428 which may be in the form of an elongated ellipse, or of sufficiently large diameter, so as to bind on needle shaft 412, when pivoting member 408 is in the deployed configuration of FIG. 21a.

When needle guard 400 is in its undeployed position, biasing member 406 is in compression. When guard 400 is slid down needle shaft 412, as soon as the needle tip clears aperture 426, the lateral force exerted by needle shaft 412 against the interior surface of aperture 426 (in reaction to the force being exerted by biasing member 406 to pivot pivoting member 408) is removed, and pivoting member 408 is free to pivot, around corner 414, to the extent that diagonal portion 418 permits. During this pivoting the inside corner edges of aperture 428 of pivoting member 408 will engage needle shaft 412, providing frictional resistance to the further sliding of needle shaft 412, relative to guard 400. Arm 422 will have sufficient width to prevent the reemergence of the needle tip out of the front of guard 400.

A still further alternative embodiment of the invention is shown in FIGS. 22a, 22b and 22c. While the carrier or loading tube is omitted from the drawings of this embodiment, it is understood that the guard of this embodiment may be initially provided with a cylindrical tubular carrier, in a manner substantially identical to that disclosed in the embodiment(s) of FIGS. 1-3. In this embodiment, needle tip guard 500 includes a housing 502 with suitable bores for passage therethrough of a needle shaft, an inner support block 504, biasing member (i.e., spring) 506, and pivoting member 508. Housing 502 is provided with a back wall 505, from which block 504 projects. Housing 502 is also surrounded by an outer shell (not shown) which may have any desired configuration, and thus which has been omitted for clarity of the illustration of the inner components. Housing 502 is configured to slidably move along needle shaft 512, and may have any desired configuration, so long as it provides an interior corner 514 to act as a fulcrum for pivoting member 508, which is rotated 90 degrees with respect to block 504, relative to the spatial relationship between block 304 and pivoting member 308, of the embodiment of FIGS. 20a and 20b. Block 504 is provided with a through bore, to permit the free sliding movement of needle shaft 512 therethrough. Housing 502 has a blind bore 516, for receiving biasing member 506. Block 504 has a removed corner portion 418, which is provided to give room for the pivoting member 508 to tilt, as shown in FIG. 22a. Pivoting member 508 has a web 520, which connects arms 522, 524. Arm 522 has an aperture 526, which is configured to let needle shaft 512 pass freely therethrough. Arm 524 is provided with aperture 528 which may be in the form of an elongated ellipse, or of sufficiently large diameter, so as to bind on needle shaft 512, when pivoting member 508 is in the deployed configuration of FIG. 22a.

When needle guard 500 is in its undeployed position, biasing member 506 is in compression. When guard 500 is slid down needle shaft 512, as soon as the needle tip clears aperture 526, the lateral force exerted by needle shaft 512 against the interior surface of aperture 526 (in reaction to the force being exerted by biasing member 506 to pivot pivoting member 508) is removed, and pivoting member 508 is free to pivot, around corner 514, to the extent that removed corner portion 518 permits. During this pivoting the inside corner edges of aperture 528 of pivoting member 508 will engage needle shaft 512, providing frictional resistance to the further sliding of needle shaft 512, relative to guard 500. Arm 522 will have sufficient width to prevent the reemergence of the needle tip out of the front of guard 500.

Another alternative embodiment of the invention is shown in FIGS. 23a, 23b and 23c. While the carrier or loading tube is omitted from the drawings of this embodiment, it is understood that the guard of this embodiment may be initially provided with a cylindrical tubular carrier, in a manner substantially identical to that disclosed in the embodiment(s) of FIGS. 1-3. Guard 600 includes housing 602, having suitable bores therethrough for passage of a needle shaft 630. Guard 600 may have an outer shell of any suitable configuration. Accordingly the shell has been omitted from the drawings for simplicity of illustration of the invention. Housing 602 is provided with a back wall 605, from which block 607 projects.

Guard 600 also includes a clamp plate 604, having clamping aperture 606; biasing member (e.g., spring) 608, which resides in blind bore 610; sliding member 612 which includes web 614, leg 616 and leg 618; and pivoting member 620, which includes legs 622 and 624 (disposed at 90 degrees to one another). Aperture 606 of clamp plate 604 is either an ellipse or circular with a diameter sufficiently large to permit ready passage of needle shaft 630, but will bind thereon, once clamp plate 604 has pivoted, as in FIG. 23b.

Leg 616 of sliding member 612 is positioned between clamp plate 604 and an inner bottom surface of void 640 of housing 602. Sliding member 612 is constrained to move vertically (relative to housing 602 in FIGS. 23a and 23b) by walls 642 and 644. In the undeployed configuration, the underface of leg 618 of sliding member 612 bears down against an upper face of leg 622 of pivoting member 620. The lower face of leg 622 of pivoting member 620 rests atop the end of ledge 626. Biasing member 608 is in compression in FIG. 23a.

In moving guard 600 to its deployed position, once tip 632 of needle shaft 312 has passed by the outer face of leg 624 of pivoting member 620, the force stored in and exerted by biasing member 608 pushes clamp plate 604 downwardly, causing clamp plate 604 to pivot about interior corner 650 of housing 602.

The downwardly moving end of clamp plate 604 in turn pushes down against leg 616 of sliding member 612. This, in turn, causes leg 618 to push down against leg 622 of pivoting member 620, causing pivoting member 620 to pivot about ledge 626, causing leg 624 to swing up, and block the path through which tip 632 has just passed, preventing re-emergence of tip 632 out of the front of housing 602. The end face of leg 618 bears against the face of leg 622 to prevent further pivoting about ledge 626. At the same time, the inner edges of aperture 606 of clamp plate 604, as described hereinabove, bind needle shaft 630 to retard removal of guard 600 off of shaft 630.

In each of the several embodiments discussed herein, the housing components, such as the outer shells and housing, may preferably be fabricated from plastics or similar materials, while the moving, sliding, bending or clamping parts herein are preferably fabricated from metallic materials, for strength, resilience, and the ability to frictionally engage, if not actually “bite into” the needle shafts of the needles with which these structures will be used.

Furthermore, in each embodiment of the invention discussed herein, the needle tip guards may be used by themselves, or more preferably they may be used in combination with a needle carrier 22 such as that shown in and described with respect to the embodiment of FIGS. 1-3, for facilitating insertion of a needle shaft into a needle tip guard, without damaging the tip of the needle.

While in each of the embodiments shown hereinabove, the housings are in the form of asymmetrical molded, stamped or machined bodies, having openings on one side only, and then insertingly received in outer shells, in alternative embodiments of the invention, the housings could be in the form of mirror-image (or nearly mirror-image) housing halves, into which the working components are mounted, and then the halves mated and affixed to one another, such as by adhesive, or sonic welding, or any other suitable method.

The foregoing description and drawings merely explain and illustrate the invention, and the invention is not limited thereto, except as those skilled in the art who have the present disclosure before them will be able to make modifications and variations therein without departing from the scope of the invention.

What is claimed is:

1. A needle tip guard for placement on the shaft of a hollow needle, for positioning over a tip region of the hollow needle, following completion of use of the hollow needle, for isolating the tip region of the hollow needle, the needle tip guard comprising:
a housing, operably configured to describe a void region and having a front end region and a rear end region;
said housing having at least one passage therethrough operably configured to receive a shaft of a hollow needle therethrough;
a resilient shield member, disposed within said void region within the housing and configured for movement between a first, undeployed position and a second deployed position,
whereupon the positioning of a hollow needle shaft through the housing extending from the first end region to the second end region, the resilient shield member is retained in its first undeployed position, in a deformed configuration with a portion of the inserted hollow needle shaft preventing movement of the shield member into the deployed configuration; in which stored force in the deformed shield member, upon movement of an inserted hollow needle shaft relative to the housing such that a tip of the hollow needle shaft is received within the void region, prompts the shield member to reorient from the first position to the second deployed position,
the resilient shield member, in the second deployed position being disposed to simultaneously preclude re-emergence of a tip of the hollow needle shaft out from the front end region of the housing, and exert a restraining force against the hollow needle shaft toward inhibiting movement of the hollow needle shaft relative to the housing.

2. The needle tip guard according to claim 1, further comprising:
a needle shaft carrier, removably inserted into the housing, and extending from the front end region through to the rear end region thereof,
the needle shaft carrier having a hollow configuration with an inner passage which is configured to insertingly a hollow needle member,
the needle shaft carrier being configured for facilitating insertion of a hollow needle shaft, and subsequent removal of the needle shaft guide member from the housing, prior to use of the hollow needle.

3. The needle tip guard according to claim 1, wherein
the shield member has a first end and a second end, the first end having an aperture operably configured to receive a hollow needle shaft therethrough,
the second end being disposed to bear against a side portion of a hollow needle shaft, when the shield member is in its first undeployed position,
the second end being disposed in a path of movement of the hollow needle shaft, when the shield member is in its second deployed position, to preclude subsequent movement of a tip of the hollow needle shaft out of the front end region of the housing.

4. The needle tip guard according to claim 3, wherein the structure surrounding the aperture in the first end of the shield member is operably configured to frictionally engage a hollow needle member extending therethrough, when the shield member is disposed in its second deployed position.

5. The needle tip guard according to claim 3, wherein the second end of the shield member is configured as a straight edge region, configured to obliquely abut a side surface of a hollow needle shaft, when the shield member is in its first position.

6. The needle tip guard according to claim 3, wherein the second end of the shield member is configured as a substantially U-shaped turned under region, a curved bight portion of which is configured to obliquely abut a side surface of a hollow needle shaft, when the shield member is in its second position.

7. The needle tip guard according to claim 1, further comprising:
an outer shell operably configured to enclose and receive the housing.

8. The needle tip guard according to claim 7, further comprising:
at least one resilient attachment member disposed proximate the rear end region of the housing and
at least one attachment member engagement region disposed proximate a rear end region of the outer shell.

9. The needle tip guard according to claim 1, further comprising:
at least one resilient attachment member disposed proximate the front end region of the housing and
at least one attachment engagement region disposed proximate a front end region of the outer shell.

10. The needle tip guard according to claim 1, further comprising:
a guide lip, extending from the second end of the shield member and disposed for sliding contact relative to a portion of the surface of a hollow needle member insertingly disposed within the housing, upon relative movement of the hollow needle member relative to the needle tip guard.

11. The needle tip guard according to claim 1, wherein
the shield member has an end configured to bear against a side portion of a hollow needle shaft, when the shield member is in its first undeployed position,
the end being configured to move into a path of movement of the hollow needle shaft, when the shield member is in its second deployed position, to preclude subsequent movement of a tip of the hollow needle shaft out of the front end region of the housing,
the void region within the housing being defined in part by an interior forward wall, along which an end of the shield member is configured to slide.

12. The needle tip guard according to claim 1, wherein the interior forward wall is straight.

13. The needle tip guard according to claim 1, wherein the interior forward wall has a first portion which is disposed at a first included angle, relative to the shaft of a hollow needle which has been inserted into the housing, which first portion is distal to the hollow needle shaft, and a second portion, proximate the hollow needle shaft, which is disposed at a second included angle, relative to the shaft of a hollow needle which has been inserted into the housing, which is less than the first included angle.

14. A needle tip guard for placement on the shaft of a hollow needle, for positioning over a tip region of the hollow needle, following completion of use of the hollow needle, for isolating the tip region of the hollow needle, the needle tip guard comprising:
a housing, operably configured to describe a void region, and having a front end region and a rear end region;
the housing having at least one passage therethrough operably configured to receive a shaft of a hollow needle therethrough;
a support block, having opposed first and second ends, and disposed within said void, and having a passage extending therethrough from the first end to the second end for receiving the shaft of a hollow needle;
a shield member, disposed within the void and configured for pivotable movement within the void between an aligned configuration and an unaligned configuration,
the shield member having a substantially rigid, C-shaped configuration, with first and second arm portions overlying the first and second ends of the support block, the first and second arm portions having apertures disposed therein, which align with the passage through the support block, when a hollow needle shaft is disposed in the passage and extending from the front end region through to the rear end region of the housing;
a biasing member, operably interconnecting the shield member, the support block and the housing, for prompting the shield member from the aligned position to the unaligned position, upon withdrawal of a tip portion of the hollow needle shaft to a position within the void between the shield member and the support block; and
a clamp plate, operably associated with the shield member, for pivotable movement therewith, to frictionally engage the hollow needle shaft, upon movement of the shield member to the unaligned position, to resist withdrawal of the hollow needle shaft from the rear end region of the housing;
the shield member, upon movement to the unaligned position, being disposed to preclude re-emergence of the tip portion of the hollow needle shaft from the front end region of the housing.

15. The needle tip guard according to claim 14, further comprising:
a needle shaft carrier, removably inserted into the housing, and extending from the front end region through to the rear end region thereof,
the needle shaft carrier having a hollow configuration with an inner passage which is configured to insertingly a hollow needle member,
the needle shaft carrier being configured for facilitating insertion of a hollow needle shaft, and subsequent removal of the needle shaft guide member from the housing, prior to use of the hollow needle.

16. A needle tip guard for placement on the shaft of a hollow needle, for positioning over a tip region of the hollow needle, following completion of use of the hollow needle, for isolating the tip region of the hollow needle, the needle tip guard comprising:
a housing, operably configured to describe a void region, and having a front end region and a rear end region;
the housing having at least one passage therethrough operably configured to receive a shaft of a hollow needle therethrough;
a support block, having opposed first and second ends, and disposed within said void, and having a passage extending therethrough from the first end to the second end for receiving the shaft of a hollow needle;
a shield member, disposed within the void and configured for pivotable movement within the void between an aligned configuration and an unaligned configuration,
the shield member having a substantially rigid, C-shaped configuration, with a planar web, and first and second arm portions extending therefrom, the first and second arm portions overlying the first and second ends of the support block, the first and second arm portions each having an aperture disposed therein, which apertures align with the passage through the support block, when a hollow needle shaft is disposed in the passage and extending from the front end region through to the rear end region of the housing;
a biasing member, operably interconnecting the shield member, the support block and the housing, for prompting the shield member from the aligned position to the unaligned position, upon withdrawal of a tip portion of the hollow needle shaft to a position within the void between the shield member and the support block;
the shield member, upon movement to the unaligned position, being disposed to preclude re-emergence of the tip portion of the hollow needle shaft from the front end region of the housing and to exert a resistive force against a side surface of the hollow needle shaft, toward inhibiting further movement of the hollow needle shaft relative to the needle tip guard.

17. The needle tip guard according to claim 16, wherein the biasing member comprises a spring member, mounted within the support block.

18. The needle tip guard according to claim 16, wherein the biasing member comprises a spring member, mounted within the housing.

19. The needle tip guard according to claim 16, further comprising:
a needle shaft carrier, removably inserted into the housing, and extending from the front end region through to the rear end region thereof,
the needle shaft carrier having a hollow configuration with an inner passage which is configured to insertingly a hollow needle member,
the needle shaft carrier being configured for facilitating insertion of a hollow needle shaft, and subsequent removal of the needle shaft guide member from the housing, prior to use of the hollow needle.

20. A needle tip guard for placement on the shaft of a hollow needle, for positioning over a tip region of the hollow needle, following completion of use of the hollow needle, for isolating the tip region of the hollow needle, the needle tip guard comprising:
a housing, operably configured to define a void region, and having a front end region and a rear end region;
the housing having at least one passage therethrough operably configured to receive a shaft of a hollow needle therethrough;
a shield member, disposed within the void and configured for pivotable movement within the void between an aligned orientation permitting placement of a hollow needle shaft through the housing, and an unaligned orientation preventing passage of a hollow needle shaft through the front end region of the housing;
an engagement member operably configured to be pivotable between a nonengagement position, when a hollow needle member is positioned in the housing extending from the front end region to the rear end region, and an engagement position, when a tip region of the hollow needle member has been withdrawn into the housing,
the engagement member, in the nonengagement position permitting the free movement of the hollow needle shaft relative to the housing, and in the engagement position exerting frictional force against the hollow needle shaft toward resisting movement of the hollow needle shaft relative to the housing;
a biasing member, operably interengaging the engagement member within the housing to prompt the engagement member toward the engagement position,
an interconnecting member, operably engaging the engagement member with the shield member, and movable between a first position and a second position, wherein movement of the tip of the hollow needle shaft past the shield member, prompting the shield member to pivot, thus permitting the interconnecting member to move to a position permitting the engagement member to move to the engagement position.

21. The needle tip guard according to claim 20, further comprising:
a needle shaft carrier, removably inserted into the housing, and extending from the front end region through to the rear end region thereof,
the needle shaft carrier having a hollow configuration with an inner passage which is configured to insertingly a hollow needle member,
the needle shaft carrier being configured for facilitating insertion of a hollow needle shaft, and subsequent removal of the needle shaft guide member from the housing, prior to use of the hollow needle.

22. A needle tip guard for placement on the shaft of a hollow needle, for positioning over a tip region of the hollow needle, following completion of use of the hollow needle, for isolating the tip region of the hollow needle, the needle tip guard comprising:
a carrier including a substantially cylindrical tube having a hollow center, said cylindrical tube capable of being slidably mounted onto a needle;
a needle guard slidably mounted onto the carrier, such that the carrier and the needle guard may be simultaneously mounted onto the needle, but wherein the carrier may be removed from both the needle guard and the needle while leaving the needle guard slidably mounted on the needle;
said needle guard including a locking shield which is prompted into a locking orientation covering the needle tip upon movement of the needle tip into the needle guard.
